(19) United States
(12) Patent Application Publication
(10) Pub. No.: US 2004/0191190 A1
(43) Pub. Date: Sep. 30, 2004

(54) Cosmetic and/or pharmaceutical preparations containing plant extracts

(76) Inventors: Gilles Pauly, Nancy (FR); Philippe Moser, Essey-les-Nancy (FR); Louis Danoux, Sauixures les Nancy (FR)

Correspondence Address: COGNIS CORPORATION, PATENT DEPARTMENT, 300 BROOKSIDE AVENUE, AMBLER, PA, 19002, US

(21) Appl. No.: 10/485,243

(22) PCT Filed: Jul. 24, 2002
(86) PCT No.: PCT/EP02/08229

(30) Foreign Application Priority Data
Aug. 2, 2001 (EP) 01402096.0

(51) Int. Cl. A61K 7/42; A61K 7/06; A61K 35/78

(52) U.S. Cl. 424/59; 424/74; 424/725

(57) ABSTRACT

The invention relates to cosmetic and/or pharmaceutical preparations containing an extract of the plant Cajanus cajan. The invention also relates to the variety of ways in which extracts of Cajanus cajan can be used in skin care and/or hair care products, for example in sunscreen products, as anti-inflammatory agents, as antioxidants and radical interceptors, as agents for combating skin ageing, as protease inhibitors, as anti-glycosylation agents and as hair care agents for improving combability. The invention further relates to a method for obtaining an extract of Cajanus cajan.

### FIELD OF THE INVENTION

This invention relates generally to cosmetology and, more particularly, to preparations containing special plant extracts and to the use of these plant extracts in cosmetic and/or pharmaceutical preparations, for example for treating the skin.

### PRIOR ART

As an organ which envelops the organism, the skin performs sealing and intervening functions with respect to the environment. There are various biochemical and biophysical systems which maintain the integrity of this exposed organ. For example, an immune system protects the skin against damage by pathogenic microorganisms; the melanin-forming system regulates pigmentation and safeguards the skin against radiation damage; a lipid system produces lipid micelles which stem the transdermal water loss; and a regulated keratin synthesis provides the mechanically resistant horny layer. The systems mentioned are based on complex chemical processes of which the course is maintained inter alia by enzymes and regulated by enzyme inhibitors. Even a slight inhibition or disinhibition of these biochemical systems is reflected in noticeable changes to the skin. However, the visible and palpable condition of the skin is a regarded as a measure of beauty, healthiness and youthfulness. Achieving this is a general objective of skin-care cosmetics.

The human skin generally reacts to exogenous, i.e. external, stress factors, such as UV radiation, ozone or other harmful substances present in the atmosphere (environmental pollution), in the form of slight or serious irritation. In particular, the skin is damaged by the oxygen radicals and nonspecific proteinases released in irritation reactions. This can have an adverse effect, for example, on the appearance or the elasticity or the barrier functions of the skin. Thus, the body's own proteases which are mobilized in excess in the event of inflammatory processes and immune reactions, such as tryptases, elastases, collagenases and cathepsins for example, attack the skin and, in particular, its structural proteins, such as collagen and elastin.

For many years, plant extracts have been used for medicinal and also for cosmetic purposes in various cultures. New plants are always being extracted and the extracts studied for their cosmetic effects in the search to find further plants with a new or different action spectrum. Many plants, whose value was not yet known and which were regarded as exotic and unimportant, are now widely used inter alia in the cosmetics field.

Today, cosmetic preparations are available to the consumer in a variety of combinations. Consumers not only expect these cosmetics to have a certain care effect or to eliminate a certain deficiency, they are also increasingly demanding products which combine several properties and thus show an improved performance spectrum. There is a particular interest in substances which both positively influence the technical properties of the cosmetic product, such as storage stability, light stability and formulatability, and at the same time represent active principles that impart, for example, caring, irritation-inhibiting, inflammation-inhibiting and/or sun protection properties to the skin and/or hair. In addition, consumers demand high dermatological compatibility and, above all, the use of natural products.

There is also a general demand for cosmetic and pharmaceutical preparations which, by virtue of their special composition, have high-quality technical properties and which, in addition, are distinguished by additional properties for the skin and hair.

There is a growing interest in care components which combine pharmaceutical activity with minimal side effects, more especially in the border areas between cosmetology and pharmacology. If these care components are incorporated in cosmetic preparations, the consumer is able conveniently to eliminate or prevent deficiency symptoms without significant effort.

### DESCRIPTION OF THE INVENTION

The problem addressed by the present invention was to provide cosmetic and/or pharmaceutical preparations that would meet the requirements for cosmetic formulations, such as storage stability and dermatological compatibility, and—besides care properties—would have, above all, improved protecting properties for human skin and/or hair, for example against UV radiation and other environmental influences, and at the same time would show preventive and curative effects on signs of ageing of the skin and could be used as anti-inflammatory agents.

Another problem addressed by the present invention was to provide preparations which would contain active components from renewable raw materials and which, at the same time, would be usable as multifunctional care components both in skin-care cosmetics and in hair care.

The present invention relates to cosmetic and/or pharmaceutical preparations containing an extract of the plant Cajanus cajan. Cosmetic and/or dermopharmaceutical preparations are preferred.

Plants in the context of the invention are understood to be both whole plants and plant parts (branches, leaves, flowers, fruit, pericarps, roots) and mixtures thereof. The extraction of leaves is preferred.

The terms “preparation” and “care preparation” are used synonymously in the present specification.

Caianus caian

The extracts to be used in accordance with the invention are obtained from the plant Cajanus cajan. This plant, which is also known as Cajanus indicus, Cytisus cajan and pigeon pea, is a 0.5 to 4 meter-tall, freely branching shrub or bush with thin roots up to 2 meters long which are also known as taproots. The trunk is up to 15 cm in diameter. The leaves appear reciprocally as trifoliate, glandulous, elliptic leaves measuring 13-13.7 cm×1.3-5.7 cm. The shrub produces yellow or red-yellow flowers in upright, loose racemes. Its densely hair-covered characteristic, often falciform legumes hold up to 8 pea-sized seeds which are white or cream-brown in color. The seeds can also be purple, mottled or black. The seeds are eaten unripened like garden peas, occasionally with the legumes, or are ground into meal after ripening. The plant can reach up to 5 years of age but, in most cases, is only cultivated as a one-year crop. The plant originates from India but is now widespread in South-East Asia and South Africa. It is mainly cultivated for its seeds which are suitable for eating. It is also grown as a green manuring and hedging plant in paddy fields. Through its extensive root system, it improves soil quality and is used as a nitrogen-fixing plant. It serves as a host for silkworms and “lac” insects.

In traditional medicine, the plant and particularly the leaves are used as a diuretic. It is used against various skin diseases. The leaves and unripe fruits are used as compresses for improving milk secretion.

Extraction

The extracts to be used in accordance with the invention may be prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Fresh plants or parts thereof are suitable as the starting material although plants and/or plant parts which may be mechanically size-reduced before extraction are normally used. Extraction of the leaves is particularly preferred. Any size reduction methods known to the expert, for example crushing with a mortar, may be used.

Preferred solvents for the extraction process are organic solvents, water (distilled or non-distilled, preferably hot water with a temperature above 80° C.) or mixtures of organic solvents and water, more particularly low molecular weight alcohols, preferably methanol, ethanol and propanol, esters, hydrocarbons, ketones or halogen-containing hydrocarbons with more or less high water contents. Extraction with water, methanol, ethanol, pentane, hexane, heptane, acetone, propylene glycols, polyethylene glycols, ethyl acetate, dichloromethane, trichloromethane and mixtures thereof is particularly preferred. Water, methanol, ethanol and mixtures thereof are particularly preferred. The extraction process is generally carried out at 20 to 100° C., preferably at 30 to 90° C. and more particularly at 50 to 90° C. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. One hour's extraction with stirring is preferred. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction processes are in the range from 3 to 20 and more particularly 10 to 17% by weight. The present invention includes the observation that the extraction conditions and the yields of the final extracts may be selected according to the desired application. In a particularly preferred embodiment, the extracts are subsequently subjected to spray drying or freeze drying.

However, the extract of the plant Cajanus cajan may also be prepared by extraction with supercritical carbon dioxide on its own or in combination with a co-solvent.

The end product obtained may also be composed of at least one fraction of a crude extract isolated and purified by chromatography, adsorption/desorption or liquid/liquid extraction. Different specifications and active substance contents can be accommodated in this way.

Cosmetic and/or pharmaceutical preparations based on the plant Cajanus cajan show surprisingly good skin-care and hair-care properties and protective properties for the skin and hair against stress and environmental influences and, at the same time, good dermatological compatibility. The preparations thus obtained are further distinguished by a high anti-oxidative capacity which, on the one hand, protects the skin against inflammatory reactions and against oxidative skin ageing processes; on the other hand, the cosmetic preparations are simultaneously protected against oxidative degradation (deterioration). The products thus obtained are also suitable for counteracting UV-induced damage to human fibroblasts and keratinocytes and, accordingly, may be used as sun protection factors in cosmetic products. They show proteinase-inhibiting properties and counteract non-enzymatic glycosylation. Both these effects also afford protection against ageing of the skin.

The extracts according to the invention have an active substance content in the extracts of 5 to 100% by weight, preferably 10 to 95% by weight and more particularly 20 to 80% by weight. In the context of the invention, the active substance content is the sum total of all the active substances present in the extract, based on the dry weight of the extract.

Active substance in the context of the invention relates to the ingredients present in the extract even if their content and identity have yet to be established by conventional methods known to the expert. Active substances in the context of the invention are also any ingredients present in the extract of which the effect is either already known or has not yet been identified by conventional methods known to the expert.

Active substance in the context of the invention relates to the percentage content of substances and auxiliaries and additives present in the preparation except for the water additionally introduced.

The quantity in which the plant extracts are used in the preparations mentioned is governed by the concentration of the individual ingredients and the manner in which the extracts are used. The plant extract is generally used in a quantity of 0.001 to 25% by weight, more particularly 0.03 to 10% by weight, 0.01 to 8% by weight, preferably 0.1 to 5% by weight and, in a particularly preferred embodiment, 1 to 3% by weight, expressed as dry weight and based on the final preparation of the cosmetic and/or pharmaceutical preparations, with the proviso that the quantities mentioned add up to 100% by weight with other auxiliaries and additives and with water.

The total content of auxiliaries and additives may be 1 to 50% by weight and is preferably 5 to 40% by weight, based on the final cosmetic and/or pharmaceutical preparations. The preparations may be produced by standard cold or hot processes but are preferably produced by the phase inversion temperature method.

Extracts

The extracts of the plant Cajanus cajan according to the invention generally contain ingredients from the group consisting of flavonoids, tannins, phytosterols, proteins, carbohydrates, phenolic acids and triterpenes. These ingredients differ in their composition according to the starting material and the extraction method selected.

Tannins in the context of the invention are tannins which can be isolated from the plant Cajanus cajan. In particular, they are polyphenols which are also known as gallotannins by virtue of their derivation from gallic acid. They are mixtures of substances of the pentadigalloyl glucose type (C₇₆H₅₂₀₄₆, MR 1701,22). They are also substances which are formed by oxidative coupling of the galloyl groups in 1,2,3,4,6-pentagalloyl-D-glucose and derivatives thereof.

Phytosterols in the context of the invention are those which can be isolated from the plant Cajanus cajan. They have a double bond at C-22 and C1 or C2 substituents at C-24. Ergosterol, stigmasterol and sitosterol, especially β-sitosterol, are particularly preferred.

Proteins in the context of the invention are any proteins which can be isolated from the plant Cajanus cajan. They make up 15 to 25% and more particularly 15 to 20% of the dry weight of the extract. Proteins are a constituent of the plant plasma and, accordingly, are found in all parts of the plant, but especially in the fruit.

Carbohydrates in the context of the invention are those which can be isolated from the plant Cajanus cajan. Such carbohydrates are preferably cellulose, glucan, inulin, agar agar, carrageenan and alginic acid. Besides cellulose, lignin is also present in the plant extract.

Phenolic acids in the context of the invention are those which can be isolated from the plant Cajanus cajan. They occur in free form or as esters or glycosides. Preferred phenolic acids are p-hydroxybenzoic acid and o-hydroxybenzoic acid, protocatechoic acid, vanillic acid, caffeic acid, p-coumaric acid, ferulic acid or salicylic acid.

Triterpenes in the context of the invention are those which can be isolated from the plant Cajanus cajan. The triterpenes according to the invention may formally be regarded as polymerization products of the hydrocarbon isoprene. The triterpenes (C30) are formed from three isoprene residues. Various polycyclic ring systems for the possible triterpenes may be derived from various folding possibilities of the three isoprene residues. The cyclization preferably yields 6-rings and—with most tetracyclic triterpenes (for example cucurbitacins) and some pentacyclic triterpenes (for example lupans)—also 5-rings. Since the 6-rings are present in the chair and tub form and the 5-rings can be flat or angled, many different skeletons are possible.

The present invention includes the observation that particularly effective cosmetic preparations are obtained through the co-operation of the ingredients of the plant extracts, particularly those mentioned above.

The present invention also relates to the various uses of the Cajanus cajan plant extracts, for example

in skin-care and/or hair-care preparations;

in skin-care preparations with a soothing, relieving and irritation-inhibiting effect on the skin, particularly for sensitive skin;

in sun protection compositions, more particularly in compositions against damage to human skin cells by UV radiation, more particularly fibroblasts and/or keratinocytes by UV-A radiation and/or UV-B radiation;

as anti-inflammatory additives;

as antioxidants or as radical traps;

as anti-rosacea agents;

as agents against hormonally and/or bacterially induced skin changes, more particularly against acne;

in care preparations against ageing of the skin for the preventive or curative treatment of signs of skin ageing;

as protease inhibitors, more particularly as MMP, collagenase and/or elastase inhibitors;

as anti-glycosylation additives, more particularly against the glycosylation of cutaneous proteins and preferably against the glycosylation of collagen;

in hair-care preparations, more particularly for improving combability.

Care Preparations

Care preparations in the context of the invention are understood to be skin-care and hair-care preparations. These care preparations include inter alia cleaning and restorative activity and UV protection. In principle, the extracts according to the invention may be used in any cosmetic products. Examples of cosmetic products and their formulations can be found in Tables 10 to 13.

The object of hair care is to maintain the natural state of freshly regrown hair for as long as possible or to restore it in the event of damage. Features of natural healthy hair include a silky sheen, low porosity, “bounce” and softness and a pleasantly smooth feel (good “handle”). The care preparations according to the invention have a smoothing effect on the hair, improve combability, reduce electrostatic charging and improve feel and sheen.

The preparations according to the invention combine excellent skin-care activity with high dermatological compatibility. In addition, they show high stability, particularly to oxidative decomposition of the products.

The present invention relates to the use of extracts of the plant Cajanus cajan in care preparations with a soothing, relieving and irritation-inhibiting effect, particularly on sensitive and/or damaged skin and/or scalp.

Sun (UV) Protection Factors

The present invention also relates to the use of the Cajanus cajan extracts in sun protection products.

Sun protection factors or UV protection factors in the context of the invention are light protection factors which are useful in protecting human skin against harmful effects of direct and indirect solar radiation. The ultraviolet radiation of the sun responsible for tanning of the skin is divided into the sections UV-C (wavelengths 200-280 nm), UV-B (280-315 nm) and UV-A (315400 nm).

The pigmenting of normal skin under the influence of solar radiation, i.e. the formation of melanins, is differently effected by UV-B and UV-A. Exposure to UV-A (long-wave UV) results in darkening of the melanins already present in the epidermis without any sign of harmful effects. It is different with so-called short-wave UV (UV-B). This promotes the formation of so-called late pigment through the reformation of melanins. However, before the (protective) pigment is formed, the skin is exposed to the unfiltered radiation which, depending on the exposure time, can lead to reddening of the skin (erythema), inflammation of the skin (sunburn) or even blisters.

Extracts of the plant Cajanus cajan are used as UV absorbers, which convert UV radiation into harmless heat, and may additionally be present in combination with other sun protection factors or UV protection factors.

These other UV protection factors are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)camphor as described in EP-B1 0693471;

4-aminobenzoic acid derivatives, preferably 4-(dimethylamino)benzoic acid-2-ethylhexyl ester, 4-(dimethylamino)benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (UVAsorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 19712033 A1 (BASF). The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or dimethicones. So-called micro- or nanopigments are preferably used in sun protection products. Micronized zinc oxide is preferably used.

In one particular embodiment of the invention, the extracts of the plant Cajanus cajan are used in preparations against damage to human skin cells by UV radiation, more particularly fibroblasts and/or keratinocytes by UV-A radiation and/or UV-B radiation, and/or as anti-inflammatory additives.

UV-A rays penetrate into the dermis where they lead to oxidative stress which is demonstrated by lipoperoxidation of the cytoplasm membranes. The lipoperoxides are degraded to malonaldialdehyde (MDA) which will crosslink many biological molecules, such as proteins and nuclein bases (enzyme inhibition or mutagenesis). The extracts of the plant Cajanus cajan according to the invention significantly reduce the level of MDA in human fibroblasts induced by UV-A rays and thus show a high capacity for reducing the harmful effects of oxidative stress on the skin.

UV-B rays initiate inflammation by activating an enzyme, namely phospholipase A2 or PLA2. This inflammation (erythema, odema) is induced by the removal of arachidonic acid from the phospholipids of the plasma membrane by the phospholipase. Arachidonic acid is the precursor of the prostaglandins which cause inflammation and cell membrane damage. The prostaglandins E2 (=PGE2) are formed by cyclooxygenase. The degree of release of the cytoplasm enzyme LDH (lactate dehydrogenase) in human keratinocytes serves as a marker for cell damage.

The extracts of the plant Cajanus cajan according to the invention reduce the effect of UV-B radiation on the number of keratinocytes and on the content of released LDH. Accordingly, the extracts have the ability to reduce cell membrane damage caused by UV-B radiation.

In principle, the extracts according to the invention may be used as anti-inflammatory additives for any cosmetic and/or pharmaceutical preparations used against inflammation of the skin and hence in skin care. The inflammation of the skin may be caused by various factors.

Antioxidants or Radical Traps

Antioxidants in the context of the invention are oxidation inhibitors which can be isolated from the plant Cajanus cajan. Antioxidants are capable of inhibiting or preventing changes caused by the effects of oxygen and other oxidative processes in the substances to be protected. The effect of antioxidants consists mainly in their acting as radical traps for the free radicals occurring during autoxidation.

Besides the use of extracts of the plant Cajanus cajan as antioxidants, other already known antioxidants may also be used. One possible use of the antioxidants, for example in cosmetic and/or pharmaceutical preparations, is their use as secondary sun protection factors because antioxidants are capable of interrupting the photochemical reaction chain which is initiated when UV rays penetrate into the skin.

Besides the plant extract according to the invention, typical examples are amino acids (for example glycine, alanine, arginine, serine, threonine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, boldin, boldo extract, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The other UV protection factors or other antioxidants may be added in quantities of 0.01 to 25, preferably 0.03 to 10 and more particularly 0.1 to 5% by weight, based on the total quantity in the preparations.

The present invention also relates to the use of Cajanus cajan extracts as anti-rosacea agents. Rosacea is the name given to chronic changes to the skin of which the cause is still not known. Its manifestations on the skin are wide-ranging. In most cases, rosacea appears as a lasting reddening of the skin, above all facial skin. It leads to roughness of the skin and to skin rash.

The present invention also relates to the use of Cajanus cajan extracts as agents against hormonally and/or bacterially induced skin changes, more particularly against acne.

Besides rosacea, they are many other unwanted changes in the skin, for example hormonally or bacterially induced changes. In the context of the invention, such changes include in particular all forms of acne.

Ageing of the Skin

The preparations and Cajanus cajan extracts according to the invention are also active against ageing of the skin and may be used for the preventive or curative treatment of signs of ageing of the skin. Care products of this type are also known as anti-ageing preparations. These signs of ageing include, for example, any type of lining or wrinkling. The treatments include the retardation of skin ageing processes. The signs of ageing can be caused by various factors. More particularly, they are attributable to UV-induced skin damage.

Protease Inhibition

The Cajanus cajan extracts according to the invention act as protease inhibitors, more particularly as MMP and/or collagenase and/or elastase inhibitors. MMPs are matrix metalloproteases which include collagenase and also elastases of a certain type. The activity of the enzymes is dependent on metal ions—in many cases Zn²⁺ ions. In the event of inflammatory processes in the skin, proteases, such as the serine protease elastase for example, or matrix metalloproteases (MMPs), such as collagenase, and another elastin-degrading elastase belonging to the MMPs, are released by the macrophages and by polymorphonuclear neutrophilic granulocytes. In addition, interstitial collagenases (also known as MMP-1s) are released in elderly people and after UV exposure.

These proteases (collagenase and the various elastases) catalyze the fragmentation and destruction of the dermal macromolecules, such as proteoglycan, collagen and elastin. They thus weaken the connective tissue and thus lead to ageing of the skin and to the effects of natural skin ageing after UV exposure.

The elastase mainly occurring belongs to the group of serine proteases. Their catalytic reaction is based on another mechanism. These proteases (collagenase and the various elastases) catalyze the fragmentation and destruction of the dermal macromolecules, such as proteoglycan, collagen and elastin and thus lead to ageing of the skin and to the effects of natural skin ageing after UV exposure.

Anti-Glycosylation

The preparations and Cajanus cajan extracts according to the invention show anti-glycosylation activity and, in particular, are active against the glycosylation of cutaneous proteins and preferably against the glycosylation of collagen. In 1981, A. Cerami (Science, 1981, 211, 491-493) described the glycosylation of proteins or non-enzymatic glysolyation as opposed to enzymatic glycosylation by glucosyl transferase and mentioned the possible role of this glycosylation in the ageing of tissue. The biochemical mechanism of this reaction is well known (Borel J. P. et al., CR biologie prospective, 145-149, 1993) and comprises two phases:

In an early phase, reducing sugars (glucose, fructose) react with the terminal or lateral amino functions of the proteins present in tissue to form so-called Schiff's bases. These compounds are then stabilized by Amadori rearrangement to the ketoamine.

In a late phase, the ketoamine functions are then oxidized in the presence of oxygen to form deoxyonose and react with other basic amino acids, such as arginine or lysine, belonging to other proteins (albumin, lipoproteins, immunoglobulin). This results in the formation of complexes which are ultimately bridged through pentosidine or 2-furoyl-1,4-imidazole cycles. The so-called AGEs (advanced glycosylation end products) are formed as complex and highly stable end products of this bridging. This late phase is very slow and irreversible.

The glycosylation of the proteins leads to the formation of inter- and intramolecular bridges in slowly renewed proteins and ultimately to the brown coloration and insolubility of these proteins. Glycosylation particularly affects the proteins in the extracellular matrices of which the renewal is slow.

In the case of the skin, the proteins damaged by glycosylation are in particular fibronectin, laminin, elastin and the various collagen types.

The AGEs lead to various complaints:

because they are bulky, the molecules which carry them have difficulty in remaining in their normal place

the glycosylated molecules lose their flexibility (tissue stiffening)

the glycosylated molecules can become more resistant to enzymes which guarantee their renewal and thus form areas of amorphous substances.

In normal skin, the glycosylated proteins are eliminated via the metabolism and the cells, more particularly through degradation by macrophages which induces re-formation of the dermis.

However, this elimination diminishes with increasing age, resulting in the accumulation of these glycosylated proteins and in accelerated and increased ageing of the dermis for which several phenomena together are responsible.

resistance to renewal proteases and a decrease in fibrillogenesis and hence in the renewal of collagen, a reduction in its filter effect in the extracellular matrix and more particularly at the dermis/epidermis boundary after fixing of foreign proteins (LDL, cholesterol, albumin) which leads to thickening,

the glycosylated proteins represent a potential source of free oxygen radicals. This phenomenon—intensified by UV-A—leads to collagen degradation,

activation of non-specific harmful proteases,

activation of macrophages and dumping of cytokinins (TNF-alpha)

finally inflammation with subsequent fibrosis and deposition of lipofuszin.

The foregoing observations are of interest for the use of substances with anti-glycosylation activity, more particularly in the control and prevention of ageing of the skin and especially the hair in the cosmetics field. The suppression of non-enzymatic glycosylation is inter alia an important objective in the prevention of hair loss.

Studies have shown that high exposure of the skin to UV radiation leads to an increase in glycosylation. Since the scalp is particularly exposed to UV radiation and since glycosylation correlates directly with the advance of hair loss, preparations which counteract glycosylation may be directly used against hair loss. If such preparations additionally act as UV/IR protection factors, as the preparations according to the invention have been shown to do, they can be said to have the desired manifold effect.

The preparations according to the invention containing Cajanus cajan extracts improve the combability of the hair and especially the combability of dry hair. This activity enables the extracts to be used in any hair care products, such as for example shampoos, hair lotions, hair rinses, hair treatments, hair setting preparations, and especially in combined preparations designed for daily application to wet or dry hair and to hair damaged by bleaching or permanent waving.

The present invention also relates to a process for the preparation of an extract of the plant Cajanus cajan, characterized in that solvents or mixtures of solvents selected from the group consisting of distilled or non-distilled water, low molecular weight alcohols, esters, hydrocarbons, ketones or halogen-containing hydrocarbons are used for extraction and the extract thus obtained is optionally dried. In a preferred embodiment of the process according to the invention, distilled water, methanol, ethanol or mixtures of water and methanol or water and ethanol are used for extraction.

The extracts according to the invention may be used in cosmetic and/or pharmaceutical preparations such as, for example, shampoos, hair lotions, foam baths, shower baths, creams, gels, lotions, sun protection products, alcohol and water/alcohol solutions, emulsions, wax/fat compounds, stick preparations, powders or ointments. These preparations may additionally contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, anti-dandruff agents, film formers, swelling agents, insect repellents, self-tanning agents, tyrosinase inhibitors (depigmenting agents), hydrotropes, solubilizers, preservatives, perfume oils, dyes and the like as further auxiliaries and additives.

Suitable surface-active auxiliaries and additives are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzene-sulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, a-olefin sulfonates, ether carboxylic acids, alkyl oligoglucosides, fatty acid glucamides, alkylamidobetaines, amphoacetals and/or protein fatty acid condensates, preferably based on wheat proteins.

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols, esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₂₋₁₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alkyl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;

mixed esters of pentaerythritol, fatty acids, citric acid and fatty alcohol and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfac-tants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine.

Finally, cationic surfactants are also suitable emulsifiers, those of the esterquat type, preferably methyl-quaternized difatty acid triethanolamine ester salts, being particularly preferred.

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol mono-esters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Metal salts of fatty acids such as, for example, magnesium, aluminium and/or zinc stearate or ricinoleate may be used as stabilizers.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grunau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

Biogenic agents in the context of the invention are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, other plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy) -propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, famesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, a-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, P-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Suitable swelling agents for aqueous phases are montmorillonites, clay minerals, Pemulen and alkyl-modified Carbopol types (Goodrich).

Suitable insect repellents are N,N-diethyl-m-toluamide, pentane-1,2-diol or Ethyl Butylacetylaminopropionate.

A suitable self-tanning agent is dihydroxyacetone. Suitable tyrosinase inhibitors which prevent the formation of melanin and are used in depigmenting agents are, for example, arbutin, ferulic acid, koji acid, coumaric acid and ascorbic acid (vitamin C).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipenta-erythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Suitable preservatives are, for example, phenoxyethanol, formal-dehyde solution, parabens, pentanediol or sorbic acid and the other classes of compounds listed in Appendix 6, Parts A and B of the Kosmetikverordnung (“Cosmetics Directive”).

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, a-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, a-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable dyes are any of the substances suitable and approved for cosmetic purposes. These dyes are normally used in concentrations of 0.001 to 0.1% by weight, based on the preparation as a whole.

### EXAMPLES

### Example 1

100 g size-reduced leaves of Cajanus cajan plants from India were placed in a glass reactor. 2 liters distilled water were then poured on. The infusion was heated to between 80 and 90° C. and extracted at that temperature with stirring for 1 hour. The mixture was then cooled to 20° C. and centrifuged for 15 mins. at a speed of 5000 G. The supernatant colloidal liquid was separated from the residue by filtration using a depth filter with a mean porosity of 0.450 μm (from Seitz of Bordeaux, France). Mannitol was added as adjuvant (1 part mannitol to 3 parts filtered supernatant) and the extract was spray-dried at a starting temperature of 185° C. and an end temperature of 80° C. The yield of dry product was 17% by weight, based on the dry weight of plants used.

### Example 2

Example 1 was repeated except that extraction was carried out with 300 g of size-reduced leaves of the plant Cajanus cajan from India in 3 liters 80% by weight aqueous methanol. The extraction process was carried out with stirring under reflux for 1 hour at boiling temperature and the extract was further processed as described. Filtration was carried out as described in Example 1. Thereafter, first the alcohol was removed under reduced pressure at 30° C. and then the water-containing residue was spray-dried as described. The extraction process was carried out on two samples or batches (batch A and batch B) of starting material. The yield of dry product amounted to 12.4% by weight for batch A and to 10.9% by weight for batch B, based on the dry weight of plants used.

### Example 3

Example 1 was repeated except that extraction was carried out with 1.9 liters 60% by volume aqueous ethanol. The extraction process was carried out over a period of 1 hour with stirring at a temperature of 50 to 65° C. and the extract was further processed as described. Filtration was carried out as described in Example 1. Thereafter, first the alcohol was removed under reduced pressure at 45° C. and then the residue was dried as described in Example 1. The yield of dry product amounted to 12.5% by weight, based on the dry weight of plants used.

### Example 4

### Activity Towards Free Radicals

In a first series of tests, the effectiveness of the extracts against oxidative stress was investigated by chemical methods. The extracts of Example 2 were used. The first test substrate selected was diphenyl picryl hydrazyl (DPPH), a purple-red colored stable radical which changes into its colorless leuco derivative on contact with radical trappers. The change of color was followed photometrically at 513 nm. The test results are set out in Table 1 (“DPPH Test”) where the inhibition of DPPH is shown in % absolute. In another test, the hydroxylation of salicylic acid by hydroxyl radicals (from the reaction of hydrogen peroxide with iron(III) ions and EDTA) was investigated as a reference system. This reaction can also be photometrically investigated because the hydroxylation product is reddish in color. The influence of the extracts on the formation of hydroxysalicylic acid was measured at an optical density of 490 nm. The results are also set out in Table 1 where inhibition is again shown in % absolute (“Salicylic Acid Test”). In a third and final test, deoxyribose was used as the test substance. In the presence of hydroxyl radicals formed from H₂O₂ in the presence of Fe²⁺ and EDTA, deoxyribose is oxidized. A pink-colored substance is formed from the oxidized form by condensation with thiobarbituric acid. The optical density was determined at 532 nm and is dependent on the content of oxidized deoxyribose. A radical-trapping substance reacts with the hydroxyl radicals formed and reduces the formation of pink-colored substances in this reaction mixture. In addition, the same reaction was carried out without EDTA to investigate complexing properties of the extract with Fe²⁺ ions. The results of the last two tests are set out in Table 2.

TABLE 1
Radical-trapping properties in % absolute (results
are the averages of two measurements)

| | | |
|---|---|---|
| | | Salicylic Acid |
| | | Test |
| | DPPH Test | Extract of |
| Concentration | Extract of Example 2 | Example 2 |

| [% by weight] | Batch A | Batch A | Batch B |
|---|---|---|---|
| Control | 0 | 0 | 0 |
| 0.001 | 30 | — | — |
| 0.01 | 68 | — | — |
| 0.03 | 90 | 33 | 11 |
| 0.1 | 92 | 61 | 43 |
| IC50 in % | 0.0068 | 0.072 | 0.115 |
| [% by weight] | | | |

TABLE 2
Radical-trapping properties with deoxyribose % absolute
(results are the averages of two measurements)

| | | |
|---|---|---|
| | EDTA | Reaction |
| | Reaction with | without EDTA |
| | Extract of | Extract of |
| Concentration | Example 2 | Example 2 |

| [% by wt.] | Batch A | Batch B | Batch A | Batch B |
|---|---|---|---|---|
| Control | 0 | 0 | 0 | 0 |
| 0.03 | 16 | 10 | 19 | 8 |
| 0.1 | 38 | 31 | 39 | 43 |
| IC50 in % | 0.14 | 0.16 | 0.14 | 0.11 |
| [% by weight] | | | | |

The results set out in Tables 1 and 2 show that the Cajanus cajan extracts used have an anti-radical effect.

In another test, the radical-trapping properties of Cajanus cajan were investigated using xanthine oxidase as the test system. Under oxidative tress, the enzyme converts purine bases, for example adenine or guanine, into uric acid with intermediate formation of oxygen radicals which are spontaneously decomposed into H₂O₂ and oxygen by Superoxid-Dismutase (SOD). These Superoxid radicals can be detected and quantitatively determined by reaction with luminol or luminol and microperoxidase and by NBT via the luminescence and the optical density at 490 nm. The luminescence yield diminishes in the presence of substances with radical-trapping properties. These results are set out in Table 3 where the inhibition is again shown in % absolute (“Luminol Test”).

TABLE 3
Test with xanthine oxidase in % absolute

| | | | Luminol + | | | |
|---|---|---|---|---|---|---|
| | Luminol | | Microperoxidase | | NBT | |
| | Extract of | | Extract of | | Extract of | |
| Concentration | Example 2 | | Exampte 2 | | Example 2 | |
| [% by weight] | Batch A | Batch B | Batch A | Batch B | Batch A | Batch B |
| Control | 0 | 0 | 0 | 0 | 0 | 0 |
| 0.001 | 45 | 6 | | | | |
| 0.01 | 92 | 71 | 0 | 8 | | |
| 0.03 | | | 57 | 51 | 21 | 22 |
| 0.1 | | | 94 | 98 | 46 | 53 |
| IC50 in % | 0.0020 | 0.0020 | 0.0275 | 0.0295 | 0.1112 | 0.0932 |

The results set out in Tables 1 to 3 show that extracts of Cajanus cajan leaves have very good radical-trapping properties. Even in a concentration of 0.001% by weight, radical-trapping properties were in evidence in the DPPH test and resulted in a 30% decoloration of the solution by comparison with the control. With a concentration of only 0.1% by weight, this test resulted in 92% decoloration. In other words, 92% of the DPPH radicals present had been “trapped”. The test with deoxyribose revealed not only radical-trapping properties, but also the ability to form complexes with Fe²⁺ ions. The luminol test again revealed very good radical-trapping properties.

### Example 5

### Toxicity

The object of this test is to determine the toxic concentration of the preparation to be studied for fibroblasts in order better to determine the most effective concentration. The effective concentration range can thus be narrowed.

Method: effects on cell growth. Human fibroblasts were inoculated with 10% by weight of fetal calf serum in a defined nutrient medium (DMEM=Dulbecco Minimum Essential Medium, a product of Life Technologie S.a.r.I.) and incubated for 24 h at 37° C. in a 5% CO₂ atmosphere. The nutrient medium containing fetal calf serum was then replaced by a nutrient medium of DMEM without fetal calf serum. Plant extract was then added to this nutrient medium in various concentrations. After the fibroblasts had been incubated for three days in the nutrient medium, growth and metabolic activity were evaluated by determining the intracellular content of ATP by Vasseur's enzymatic luminescence method (Journal Francais Hydrologie, 1981, 9, 149-156) and the cell protein content by Bradford's method (Anal. Biochem., 1976, 72, 28-254).

Determining the cell protein content provides an indication of the number of macromolecules, such as enzymes, collagen, elastin or other dermal macromolecules, which is required for forming connective tissue. The ATP content of a cell is important for many enzymes whose activity is dependent upon this energy carrier.

The lethal dose of the Cajanus cajan extract at which 50% of the fibroblasts studied were no longer viable was determined (LD 50). Up to a concentration of 0.005% by weight, the Cajanus cajan extract does not have a toxic effect on the human fibroblasts.

### Example 6

### Cell Protecting Effect Against UV-A on Human Fibroblasts Cultivated In Vitro

Background: UV-A rays penetrate into the dermis where they lead to oxidative stress which is demonstrated by lipoperoxidation of the cytoplasm membranes.

The lipoperoxides are degraded to malonaldialdehyde which will crosslink many biological molecules, such as proteins and nuclein bases (enzyme inhibition or mutagenesis).

Method: To carry out these tests, a defined culture medium (DMEM) containing the fibroblasts is inoculated with foetal calf serum and added to the plant extract (in the defined medium containing 10% foetal calf serum) 72 hours after inoculation.

After incubation for 48 hours at 37° C./5% CO₂, the culture medium was replaced by saline solution (physiological NaCl solution) and the fibroblasts were exposed to a dose of UV-A (365 nm, 15 J/cm²; tubes: MAZDA FLUOR TFWN40).

After the exposure to UV-A, the MDA level (malonaldialdehyde level) in the supernatant sodium chloride solution was quantitatively determined by reaction with thiobarbituric acid. The protein content was determined by Bradford's method.

TABLE 4
Quantification of malonaldialdehyde in fibroblasts (results in %,
based on the control, average value of 2 tests each repeated 3 times)

| | MDA content | Protein content |
|---|---|---|
| Concentration | [% versus control] | [% versus control] |
| [% by weight] | Extract of Example 1 | Extract of Example 1 |
| Control without UV | 0 | 100 |
| UV-A (365 nm) | 100 | 102 |
| UV-A + extract 0.1% | 76 | 138 |
| UV-A + extract 0.3% | 46 | — |

| | MDA content | | Protein content | |
|---|---|---|---|---|
| | [% versus control] | | [% versus control] | |
| | Extract of | | Extract of | |
| Concentration | Example 2 | | Example 2 | |
| [% by weight] | Batch A | Batch B | Batch A | Batch B |
| Control without UV-A | 0 | 0 | 100 | 100 |
| Control with UV-A | 100 | 100 | 100 | 97 |
| UV-A + extract 0.002% | 66 | 74 | 101 | 103 |
| UV-A + extract 0.005% | 60 | 50 | 93 | 110 |

| | MDA content | Protein content |
|---|---|---|
| Concentration | [% versus control] | [% versus control] |
| [% by weight] | Extract of Example 3 | Extract of Example 3 |
| Control without UV | 0 | 100 |
| UV-A (365 nm) | 100 | 102 |
| UV-A + extract 0.1% | 51 | 120 |
| UV-A + extract 0.03% | 43 | 123 |

The results set out in Table 4 show that the extracts of the plant Cajanus cajan significantly reduce the level of MDA in human fibroblasts induced by UV-A rays. These results reflect a high capacity on the part of Cajanus cajan extracts to reduce harmful effects of oxidative stress on the skin. The protein content again illustrates the nontoxic effect of the extract.

### Example 7

### Cell Protecting Effect Against UV-B on Human Keratinocytes Cultivated In Vitro

Background: UV-B rays cause inflammation (erythema, odema) by activating an enzyme, namely phospholipase A2 or PLA2, which removes arachidonic acid from the phospholipids of the plasma membrane. Arachidonic acid is the precursor of the prostaglandins which cause inflammation and cell membrane damage; the prostaglandins E2 (=PGE2) are formed by cyclooxygenase.

Method: The effect of UV-B radiation was investigated in vitro in keratinocytes by determining the release of the cytoplasm enzyme LDH (lactate dehydrogenase). This enzyme serves as a marker for cell damage.

To carry out the tests, a defined medium (DMEM) containing foetal calf serum was inoculated with the keratinocytes and the plant extract (diluted with saline solution) was added 72 hours after the inoculation. The keratinocytes were then exposed to a dose of UV-B (30 mJ/cm²—tubes: DUKE GL40E).

After incubation for another day at 37° C./5% CO₂, the LDH content in the supernatant was determined. The LDH (lactate dehydrogenase) content was determined by an enzyme reaction (kit used to determine LDH levels from Roche). The PGE2 content was determined by the ELISA Test (ELISA Kit from Roche). The number of adhering keratinocytes was determined (after trypsin treatment) with a particle counter.

TABLE 5
Cell protecting effect of the Cajanus cajan extracts against
UV-B rays; results in % based on the control, average value
of 2 tests each repeated twice.

| Extract of Example 1 | Number of | Content of LDH | |
|---|---|---|---|
| [% by weight] | keratinocytes | released | PGE2 content |
| Control without UV | 100 | 0 | 0 |
| UV-B (315 nm) | 38 | 100 | 100 |
| UV-B + extract 0.03% | 45 | 77 | — |
| UV-B + extract 0.1% | 89 | 27 | 7 |
| UV-B + extract 0.3% | 134 | 4 | 2 |

| | | | Content of | | | |
|---|---|---|---|---|---|---|
| Extract of | Number of | | LDH | | | |
| Example 2 | keratinocytes | | released | | PGE2 content | |
| [% by weight] | Batch A | Batch B | Batch A | Batch B | Batch A | Batch B |
| Control without | 100 | 100 | 0 | 0 | 0 | 0 |
| UV | | | | | | |
| UV-B | 54 | 47 | 100 | 100 | 100 | 100 |
| (315 nm) | | | | | | |
| UV-B + | 72 | 76 | 66 | 22 | 61 | 50 |
| extract 0.002% | | | | | | |
| UV-B + | 91 | 147 | 38 | 0 | 36 | 7 |
| extract 0.005% | | | | | | |

| Extract of Example 3 | Number of | Content of LDH | |
|---|---|---|---|
| [% by weight] | keratinocytes | released | PGE2 content |
| Control without UV | 100 | 0 | 0 |
| UV-B (315 nm) | 38 | 100 | 100 |
| UV-B + extract | 47 | 70 | — |
| 0.003% | | | |
| UV-B + extract 0.01% | 86 | 29 | 21 |
| UV-B + extract 0.03% | 148 | 0 | 10 |

The results of these tests show that the extracts of the plant Cajanus cajan positively influence the effect of UV-B radiation on the number of keratinocytes, on the content of LDH released and on the PGE2 content. Accordingly, the described extracts have the ability to reduce cell membrane damage caused by UV-B radiation.

### Example 8

### Anti-Protease

In the event of inflammatory processes in the skin, proteases, such as the serine protease elastase for example, or matrix metalloproteases (MMPs), such as collagenase, and another elastin-degrading elastase belonging to the MMPs, are released by the macrophages and by polymorphonuclear neutrophilic granulocytes. In addition, interstitial collagenases (also known as MMP-1s) are released in elderly people and after UV exposure.

The proteases (collagenase and the various elastases) catalyze the fragmentation and destruction of the dermal macromolecules, such as proteoglycan, collagen and elastin, and thus lead to ageing of the skin and to the effects of natural skin ageing after UV exposure.

8a: Inhibition of Elastase Activity

Serine proteases, such as a type of elastase for example, degrade elastin, proteoglycans and collagen and thus weaken the connective tissue. The following test was conducted to investigate the inhibiting properties of the extract of Example 2 on a chromogenic synthetic substrate (obtained from SIGMA). The incubation time was 30 mins. at room temperature. The inhibition was followed photometrically at 410 nm; 1′α1-antitrypsin was used as positive standard. The results are set out in Table 6.

TABLE 6
Inhibition of elastase (results in % inhibition)

| | |
|---|---|
| | Extract of |
| Concentration | Example 2 |

| [% by weight] | Batch A | Batch B |
|---|---|---|
| 0.15 | 50 | — |
| 0.3 | 92 | 35 |

The extract is thus active against the serine protease elastase. This activity leads to its possible use against effects of skin ageing which are attributable to increased activity of the serine protease elastase.

8b. Inhibition of Collagenase

After exposure to the sun, dermal fibroblasts of elderly people pour out collagenases—also known as matrix metalloprotease-1 (MMP-1). Collagenase from Clostridium histolyticum, which had been marked with chromogenic synthetic substrate FALGPA, was investigated in a first test. The incubation time was 30 mins. at room temperature. The hydrolysis of collagen was photometrically determined at 324 nm. Cystein or EDTA was used as a comparison substance.

TABLE 7
Inhibition of collagenase (results in % inhibition)

| | |
|---|---|
| | Extract of |
| Concentration | Example 2 |

| [% by weight] | Batch A | Batch B |
|---|---|---|
| 0.23 | — | 50 |
| 0.3 | 27 | 78 |

The results demonstrate the inhibiting effect of the Cajanus cajan extracts against MMP collagenase. This activity leads to their use against effects of skin ageing attributable to degradation of the dermal macromolecules collagen.

### Example 9

### Inhibition of the Glycosylation of Collagen

To show that the Cajanus cajan extracts inhibit the non-enzymatic glycosylation of macromolecules, type I collagen was treated with glucose and the extracts over a period of 21 days at 45° C. The suspension was then centrifuged and the content of Schiffs bases in the supernatant liquid was determined by fluorescence measurement at 430 nm. The results are set out in Table 7. The figures are based on the control as standard (without extract and without glucose).

TABLE 8
Yield of Schiff's bases

| | | Extract of | | |
|---|---|---|---|---|
| Concentration | Extract of | Example 2 | | Extract of |
| [% by weight] | Example 1 | Batch A | Batch B | Example 3 |
| Control without glucose | 22.9 | 3.6 | 38.4 | 22.9 |
| Control with glucose | 89.5 | 100 | 101.8 | 89.5 |
| Glucose + extract 0.01% | — | 91.3 | 89.7 | — |
| by weight | | | | |
| Glucose + extract 0.1% | — | 25.8 | 52.1 | — |
| by weight | | | | |
| Glucose + extract 0.3% | 62.6 | | | 77.6 |
| by weight | | | | |
| Glucose + extract 1% | 55 | | | 41.3 |
| by weight | | | | |
| Glucose + extract 3% | 15.6 | | | 4.7 |
| by weight | | | | |

The results reflect a reducing activity on the non-enzymatic glycosylation of collagen and hence a reducing effect on the ageing of the dermis.

### Example 10

### Sensory Activity on Human Hair

The modification of sensory properties of human hair after treatment with extracts of the plant Cajanus cajan was evaluated on standardized hair tresses (length 20 cm, weight 5 g). The samples of the plant extracts were incorporated in a shampoo and tested in a concentration of 1.5% by weight. Combability was tested on the dried hair before and after treatment with the shampoo. Before the determination of combability and before treatment with the shampoo, the hair tresses were washed with aqueous sodium lauryl sulfate solution (15% by weight).

To determine combability, the hair tresses were fixed at their upper ends and the force which has to be expended for combing was measured in a standardized apparatus in air air-conditioned atmosphere by drawing the comb through the tress at a uniform speed.

As the standard, combability was determined on dry hair tresses which had been stored overnight in an air-conditioned housing at 22° C./40% relative air humidity. Three combability tests were carried out on the same hair tress.

The treatment of the hair tresses was carried out for 3 mins. with 3 ml of shampoo containing the Cajanus cajan extract. The hair tress was then rinsed with water for 1 minute. The hair tress thus treated was also stored overnight in the air-conditioned housing at 22° C./40% relative air humidity and subjected three times to the same combability tests.

The highest force which had to be expended during combing and the total combing work, as expressed by the average value of the force measured in the particular combability tests, were both determined.

TABLE 9
Determination of the force expended in combability tests

| | |
|---|---|
| Test 1 | Test 2 |

| | | Extract of | | Extract of |
|---|---|---|---|---|
| | | Example 2 | | Example 2 |
| | Placebo | Batch A | Placebo | Batch B |
| Highest force | +8 | −28 | +11 | −34 |
| Combing work | +18 | −35 | 0 | −37 |

The Cajanus cajan extracts are suitable for hair care because they improve the combability of dry hair.

Various cosmetic products or cosmetic preparations containing an extract of the plant Cajanus cajan are described in the following as practical embodiments of the invention.

A number of Formulation Examples are set out in the following Tables.

### Example 10

### Exemplary Formulations of Cosmetic Preparations Containing Extracts of the Plant Cajanus cajan

The Cajanus cajan extracts obtained in accordance with Examples 1 to 3 were used in the following formulations K1 to K21 and 1 to 40 according to the invention. Unless otherwise specifically stated, any extract of Examples 1 to 3 may be used. The cosmetic preparations thus produced showed very good skin care properties in relation to comparison formulations C1, C2 and C3 coupled with good dermatological compatibility. In addition, the preparations according to the invention are stable to oxidative decomposition.

TABLE 10
Soft cream formulations K1 to K7
(All quantities in % by weight, based
on the cosmetic preparation)

| INCI name | K1 | K2 | K3 | K4 | K5 | K6 | K7 | C1 |
|---|---|---|---|---|---|---|---|---|
| Glyceryl Stearate (and) | 8.0 | 8.0 | 8.0 | 8.0 | 8.0 | 8.0 | 8.0 | 8.0 |
| Ceteareth-12/20 (and) | | | | | | | | |
| Cetearyl Alcohol (and) | | | | | | | | |
| Cetyl Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 3.0 | 3.0 | 3.0 | 3.0 | 3.0 | 3.0 | 3.0 | 3.0 |
| Glycerin (86% by weight) | 3.0 | 3.0 | 3.0 | 3.0 | 3.0 | 3.0 | 3.0 | 3.0 |
| Cajanus cajan extracts | 0.5 | 0.5 | 0.5 | 0.5 | 0.5 | 0.5 | 0.5 | — |
| (Examples 1-3) | | | | | | | | |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonuleic acid¹⁾ | | | | | | 0.5 | | |
| Panthenol | | | | | | | 0.5 | |

| | |
|---|---|
| Water | to 100 |

TABLE 11
Night cream formulations K8 to K14
(All quantities in % by weight, based
on the cosmetic preparation)

| INCI name | K8 | K9 | K10 | K11 | K12 | K13 | K14 | C2 |
|---|---|---|---|---|---|---|---|---|
| Polyglyceryl-2 Dipolyhydroxystearate | 4.0 | 4.0 | 4.0 | 4.0 | 4.0 | 4.0 | 4.0 | 5.0 |
| Polyglyceryl-3 Diisostearate | 2.0 | 2.0 | 2.0 | 2.0 | 2.0 | 2.0 | 2.0 | 2.0 |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 8.0 | 8.0 | 8.0 | 8.0 | 8.0 | 8.0 | 8.0 | 8.0 |
| Dicaprylyl Ether | 5.0 | 5.0 | 5.0 | 5.0 | 5.0 | 5.0 | 5.0 | 5.0 |
| Magnesium sulfate | 1.0 | 1.0 | 1.0 | 1.0 | 1.0 | 1.0 | 1.0 | 1.0 |
| Glycerin (86% by weight) | 5.0 | 5.0 | 5.0 | 5.0 | 5.0 | 5.0 | 5.0 | 5.0 |
| Cajanus cajan extract | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 12
W/O body lotion formulations K15 to K21.
(All quantities in % by weight, based
on the cosmetic preparation)

| INCI name | K15 | K16 | K17 | K18 | K19 | K20 | K21 | C3 |
|---|---|---|---|---|---|---|---|---|
| PEG-7 Hydrogenated Castor Oil | 7.0 | 7.0 | 7.0 | 7.0 | 7.0 | 7.0 | 7.0 | 7.0 |
| Decyl Oleate | 7.0 | 7.0 | 7.0 | 7.0 | 7.0 | 7.0 | 7.0 | 7.0 |
| Cetearyl Isononanoate | 7.0 | 7.0 | 7.0 | 7-0 | 7.0 | 7.0 | 7.0 | 7.0 |
| Glycerin (86% by weight) | 5.0 | 5.0 | 5.0 | 5.0 | 5.0 | 5.0 | 5.0 | 5.0 |
| MgSO₄.7H₂O | 1.0 | 1.0 | 1.0 | 1.0 | 1.0 | 1.0 | 1.0 | 1.0 |
| Cajanus cajan extract | 1.5 | 1.5 | 1.5 | 1.5 | 1.5 | 1.5 | 1.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | 0.5 | | | | | | |
| Panthenol | | | 0.5 | | | | | |

| | |
|---|---|
| Water | to 100 |

¹⁾Deoxyribonucleic acid: molecular weight ca. 70,000, purity (determined by spectrophotometric measurement of absorption at 260 nm and 280 nm): at least 1.7

TABLE 13
Formulations for conditioners I
Cosmetic preparations conditioner (water,
preservative to 100% by weight)

| Composition (INCI) | 1 | 2 | 3 | 4 | 5 | 6 |
|---|---|---|---|---|---|---|
| Dehyquart ® A | 4.0 | 4.0 | | | 3.0 | |
| Cetrimonium Chloride | | | | | | |
| Dehyquart L ® 80 | | | 1.2 | 1.2 | | 1.0 |
| Dococoylmethylethoxymonium | | | | | | |
| Methosulfate (and) Propyleneglycol | | | | | | |
| Eumulgin ® B2 | 0.8 | | — | 0.8 | — | 1.0 |
| Ceteareth-20 | | | | | | |
| Eumulgin ® VL 75 | — | 2.0 | 2.0 | — | 0.8 | — |
| Lauryl Glucoside (and) Polyglyceryl-2 | | | | | | |
| Polyhydroxystearate (and) Glycerin | | | | | | |
| Lanette ® O | 3.0 | 3.0 | 3.0 | 3.0 | 3.0 | 3.0 |
| Cetearyl Alcohol | | | | | | |
| Cutina ® GMS | — | 0.5 | — | 0.5 | — | 1.0 |
| Glyceryl Stearate | | | | | | |
| Lamesoft ® PO 65 | | — | 3.0 | — | — | 3.0 |
| Coco-Glucoside (and) Gyceryl Oleate | | | | | | |
| Cetiol ® J 600 | — | 0.5 | — | 1.0 | — | 1.0 |
| Oleyl Erucate | | | | | | |
| Eutanol ® G | — | — | 1.0 | — | — | 1.0 |
| Octyldodecanol | | | | | | |
| Nutrilan ® Keratin W | 5.0 | — | — | 2.0 | — | — |
| Hydrolyzed Keratin | | | | | | |
| Generol ® 122 N | — | — | — | — | 1.0 | 1.0 |
| Soya Sterol | | | | | | |
| Cajanus cajan extract | 1.0 | 1.0 | 1.0 | 1.0 | 1.0 | 1.0 |
| Copherol ® 12250 | — | — | 0.1 | 0.1 | — | — |
| Tocopherol Acetate | | | | | | |

(1-4) Hair rinse,
(5-6) hair treatment

TABLE 13
Formulations for conditioners II
Cosmetic preparations conditioners (water,
preservative to 100% by weight)

| Composition (INCI) | 7 | 8 | 9 | 10 |
|---|---|---|---|---|
| Texapon ® NSO | 38.0 | 38.0 | 25.0 | — |
| Sodium Laureth Sulfate | | | | |
| Texapon ® SB 3 | — | — | 10.0 | — |
| Disodium Laureth Sulfosuccinate | | | | |
| Plantacare ® 818 | 7.0 | 7.0 | 6.0 | — |
| Coco Glucosides | | | | |
| Plantacare ® PS 10 | — | — | — | 20.0 |
| Sodium Laureth Sulfate (and) | | | | |
| Coco Glucosides | | | | |
| Dehyton ® PK 45 | — | — | 10.0 | — |
| Cocamidopropyl Betaine | | | | |
| Lamesoft ® PO 65 | 3.0 | | | 4.0 |
| Coco-Glucoside (and) Glyceryl Oleate | | | | |
| Lamesoft ® LMG | — | 5.0 | — | — |
| Glyceryl Laurate (and) Potassium | | | | |
| Cocoyl Hydrolyzed Collagen | | | | |
| Euperlan ® PK 3000 AM | — | 3.0 | 5.0 | 5.0 |
| Glycol Distearate (and) Laureth-4 (and) | | | | |
| Cocamidopropyl Betaine | | | | |
| Cajanus cajan extract | 1.0 | 1.0 | 1.0 | 1.0 |
| Arlypon ® F | 3.0 | 3.0 | 1.0 | — |
| Laureth-2 | | | | |
| Sodium Chloride | — | 1.5 | — | 1.5 |

(7-8) shower bath,
(9) shower gel,
(10) wash lotion

TABLE 13
Cosmetic preparations shampoo (water,
preservative to 100% by weight)

| Composition (INCI) | 15 | 16 | 17 | 18 | 19 | 20 |
|---|---|---|---|---|---|---|
| Texapon ® NSO | 30.0 | | | 30.0 | 25.0 | |
| Sodium Laureth Sulfate | | | | | | |
| Texapon ® K 14 S | | 30.0 | | | | 30.0 |
| Sodium Myreth Sulfate | | | | | | |
| Texapon ® SB 3 | | 10.0 | | | | |
| Disodium Laureth | | | | | | |
| Sulfosuccinate | | | | | | |
| Plantacare ® 818 | 4.0 | | | | | |
| Coco Glucosides | | | | | | |
| Plantacare ® 2000 | | 4.0 | | | | |
| Decyl Glucoside | | | | | | |
| Plantacare ® PS 10 | | | 20.0 | | | |
| Sodium Laureth Sulfate | | | | | | |
| (and) Coco Glucosides | | | | | | |
| Dehyton ® PK 45 | 5.0 | | | 10.0 | | 10.0 |
| Cocamidopropyl Betaine | | | | | | |
| Gluadin ® WK | | | | | 8.0 | |
| Sodium Cocyl Hydrolyzed | | | | | | |
| Wheat Protein | | | | | | |
| Lamesoft ® PO 65 | — | — | — | — | 2.0 | 2.0 |
| Coco-Glucoside (and) | | | | | | |
| Glyceryl Oleate | | | | | | |
| Nutrilan ® Keratin W | 5.0 | — | — | — | | — |
| Hydrolyzed Keratin | | | | | | |
| Gluadin ® W 40 | — | 2.0 | — | 2.0 | — | — |
| Hydrolyzed Wheat Protein | | | | | | |
| Euperlan ® PK 3000 AM | — | — | — | 3.0 | 3.0 | — |
| Glycol Distearate (and) | | | | | | |
| Laureth-4 (and) | | | | | | |
| Cocamidopropyl Betaine | | | | | | |
| Panthenol | — | — | — | — | — | 0.2 |
| Cajanus cajan extract | 1.0 | 1.0 | 1.0 | 1.0 | 1.0 | 1.0 |
| Arlypon ® F | 1.5 | — | — | — | — | — |
| Laureth-2 | | | | | | |
| Sodium Chloride | — | 1.6 | 2.0 | 2.2 | — | 3.0 |

TABLE 13
Cosmetic preparations “2-in-1” shower bath
(water, preservative to 100% by weight)

| Composition (INCI) | 11 | 12 | 13 | 14 |
|---|---|---|---|---|
| Texapon ® NSO | 30.0 | 25.0 | | 25.0 |
| Sodium Laureth Sulfate | | | | |
| Plantacare ® 818 | | | | 8.0 |
| Coco Glucosides | | | | |
| Plantacare ® 2000 | | 8.0 | | |
| Decyl Glucoside | | | | |
| Plantacare ® PS 10 | | | 20.0 | |
| Sodium Laureth Sulfate (and) | | | | |
| Coco Glucosides | | | | |
| Dehyton ® PK 45 | | 10.0 | 10.0 | |
| Cacamidopropyl Betaine | | | | |
| Lamesoft ® PO 65 | 5.0 | | | |
| Coco-Glucoside (and) Glyceryl Oleate | | | | |
| Lamesoft ® LMG | | 5.0 | 5.0 | |
| Glyceryl Laurate (and) Potassium Cocoyl | | | | |
| Hydrolyzed Collagen | | | | |
| Gluadin ® WQ | 3.0 | | | |
| Laurdimonium Hydroxypropyl | | | | |
| Hydrolyzed Wheat Protein | | | | |
| Gluadin ® WK | | | | |
| Sodium Cocoyl Hydrolyzed Wheat Protein | | | | |
| Euperlan ® PK 3000 AM | 5.0 | 3.0 | 4.0 | — |
| Glycol Distearate (and) Laureth-4 (and) | | | | |
| Cocamidopropyl Betaine | | | | |
| Panthenol | 0.5 | — | — | 0.5 |
| Cajanus cajan extract | 1.0 | 1.0 | 1.0 | 1.0 |
| Arlypon ® F | 2.6 | 1.6 | — | 1.0 |
| Laureth-2 | | | | |
| Sodium Chloride | — | — | — | — |

TABLE 13
Cosmetic preparations foam bath (water,
preservative to 100% by weight)

| Composition (INCI) | 21 | 22 | 23 | 24 | 25 |
|---|---|---|---|---|---|
| Texapon ® NSO | — | 30.0 | 30.0 | — | 25.0 |
| Sodium Laureth Sulfate | | | | | |
| Plantacare ® 818 | — | 10.0 | — | — | 20.0 |
| Coco Glucosides | | | | | |
| Plantacare ® PS 10 | 22.0 | — | 5.0 | 22.0 | — |
| Sodium Laureth Sulfate (and) | | | | | |
| Coco Glucosides | | | | | |
| Dehyton ® PK 45 | 15.0 | 10.0 | 15.0 | 15.0 | 15.0 |
| Cocamidopropyl Betaine | | | | | |
| Monomuls ® 90-O 18 | 0.5 | | | | |
| Glyceryl Oleate | | | | | |
| Lamesoft ® PO 65 | | 3.0 | | 3.0 | 2.0 |
| Coco-Glucoside (and) | | | | | |
| Glyceryl Oleate | | | | | |
| Cetiol ® HE | | | 2.0 | | 2.0 |
| PEG-7 Glyceryl Cocoate | | | | | |
| Nutrilan ® I-50 | 5.0 | | | | |
| Hydrolyzed Collagen | | | | | |
| Gluadin ® W 40 | | 5.0 | | 5.0 | |
| Hydrolyzed Wheat Gluten | | | | | |
| Gluadin ® WK | | | | 7.0 | |
| Sodium Cocoyl Hydrolyzed | | | | | |
| Wheat Protein | | | | | |
| Euperlan ® PK 3000 AM | 5.0 | — | — | 5.0 | — |
| Glycol Distearate (and) Laureth-4 | | | | | |
| (and) Cocamidopropyl Betaine | | | | | |
| Arlypon ® F | | | 1.0 | | |
| Laureth-2 | | | | | |
| Sodium Chloride | 1.0 | | 1.0 | | 2.0 |
| Cajanus cajan extract | 1.0 | 1.0 | 1.0 | 1.0 | 1.0 |

TABLE 13
Cosmetic preparations (water, preservative to 100% by weight)

| Composition (INCI) | 31 | 32 | 33 | 34 | 35 | 36 | 37 | 38 | 39 | 40 |
|---|---|---|---|---|---|---|---|---|---|---|
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 Methylglucose Distearate | | | | | | | | | | |
| Eumulgin VL 75 | — | — | — | — | — | 3.5 | — | — | 2.5 | — |
| Polyglyceryl-2 Dipolyhydroxystearate (and) | | | | | | | | | | |
| Lauryl Glucoside (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Panthenol/Bisabolol | 1.2 | 1.2 | 1.2 | 1.2 | 1.2 | 1.2 | 1.2 | 1.2 | 1.2 | 1.2 |
| Cajanus cajan extract | 1.0 | 1.0 | 1.0 | 1.0 | 1.0 | 1.0 | 1.0 | 1.0 | 1.0 | 1.0 |
| Copherol ® F 1300 | 0.5 | 1.0 | 1.0 | 2.0 | 1.0 | 1.0 | 1.0 | 2.0 | 0.5 | 2.0 |
| Tocopherol/Tocopheryl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerol (86% by weight) | 5.0 | 5.0 | 5.0 | 5.0 | 5.0 | 5.0 | 5.0 | 5.0 | 5.0 | 5.0 |

(31) w/o sun protection cream, (32-34) w/o sun protection lotion, (35,38,40) o/w sun protection lotion, (36,37,39) o/w sun protection cream All substances with the registered trade mark symbol® used and listed in Tables 10 to 13 are marks and products of the COGNIS Group.

1. Cosmetic and/or pharmaceutical preparations containing an extract of the plant Cajanus cajan.

2. Preparations as claimed in claim 1, characterized in that they contain the plant extract in quantities of 0.001 to 25% by weight, expressed as dry weight and based on the total quantity of the preparations, with the proviso that the quantities mentioned add up to 100% by weight with water and, optionally, other auxiliaries and additives.

3. Preparations as claimed in claim 1, characterized in that they contain substances selected from the group consisting of flavonoids, tannins, phytosterols, proteins, carbohydrates, phenolic acids and triterpenes.

4. The use of extracts of the plant Cajanus cajan in skin-care and/or hair-care preparations.

5. The use of extracts of the plant Cajanus cajan in skin-care preparations with a soothing, relieving and irritation-inhibiting effect on the skin, particularly for sensitive skin.

6. The use of extracts of the plant Cajanus cajan in sun protection compositions.

7. The use claimed in claim 6 in compositions against damage to human skin cells by UV radiation, more particularly fibroblasts and/or keratinocytes by UV-A radiation and/or UV-B radiation.

8. The use of extracts of the plant Cajanus cajan as anti-inflammatory additives.

9. The use of extracts of the plant Cajanus cajan as antioxidants or as radical traps.

10. The use of extracts of the plant Cajanus cajan as anti-rosacea agents.

11. The use of extracts of the plant Cajanus cajan as agents against hormonally and/or bacterially induced skin changes, more particularly against acne.

12. The use of extracts of the plant Cajanus cajan in care preparations against ageing of the skin for the preventive or curative treatment of signs of skin ageing.

13. The use of extracts of leaves of the plant Cajanus cajan as protease inhibitors, more particularly as MMP, collagenase and/or elastase inhibitors.

14. The use of extracts of leaves of the plant Cajanus cajan as anti-glycosylation additives, more particularly against the glycosylation of cutaneous proteins and preferably against the glycosylation of collagen.

15. The use of extracts of leaves of the plant Cajanus cajan in hair-care preparations, more particularly for improving combability.

16. A process for the preparation of an extract of the plant Cajanus cajan, characterized in that solvents or mixtures of solvents selected from the group consisting of distilled or non-distilled water, low molecular weight alcohols, esters, hydrocarbons, ketones or halogen-containing hydrocarbons are used for extraction and the extract thus obtained is optionally dried.

17. A process for the preparation of an extract of the plant Cajanus cajan, characterized in that supercritical carbon dioxide on its own or in combination with a co-solvent is used for extraction.

18. A process for the preparation of an extract of the plant Cajanus cajan, characterized in that the extract is obtained from at least one fraction of a crude extract isolated and purified by chromatography, adsorption/desorption or liquid/liquid extraction.
